(19) United States
(12) Patent Application Publication
(10) Pub. No.: US 2011/0137390 A1
(43) Pub. Date: Jun. 9, 2011

(54) SYSTEM AND METHOD FOR PROTECTING IMPLANTED MEDICAL DEVICES FROM INTERFERING RADIATED FIELDS

(76) Inventor: Gerard J. Hill, Champlin, MN (US)

(21) Appl. No.: 12/961,584

(22) Filed: Dec. 7, 2010

Related U.S. Application Data
(60) Provisional application No. 61/267,736, filed on Dec. 8, 2009.

(51) Int. Cl. A61N 1/00

(52) U.S. Cl. 607/116

(57) ABSTRACT

An implantable medical system includes an implantable medical device (IMD) and at least one lead coupled to the IMD at a proximal end to anatomic tissue of a patient at a distal end. According to various embodiments, a piezoelectric transformer permits the transmission of intended signals, such as physiologic signals or therapy signals, while preventing an interfering signal from being transmitted toward the circuitry for generating therapy or causing heating of the anatomical tissue.

This application claims the benefit of U.S. Provisional Application No. 61/267,736, filed on Dec. 8, 2009, the content of which is incorporated herein by reference in its entirety.

### FIELD

The present disclosure relates to implantable medical devices (IMDs), in particular to a system and method for use of a piezoelectric transformer to protect IMDs from interfering radiated fields such as present during a magnetic resonance imaging procedure.

### BACKGROUND

The statements in this section merely provide background information related to the present disclosure and may not constitute prior art.

The human anatomy includes many types of tissue that can either voluntarily or involuntarily, perform certain functions. However, after disease or injury, certain tissues may no longer operate within general anatomical norms. For example, after disease, injury, age, or combinations thereof, the heart muscle may begin to experience certain failures or deficiencies. Some of these failures or deficiencies can be corrected or treated with implantable medical devices (IMDs). These devices can include implantable pulse generator (IPG) devices, pacemakers, implantable cardioverter-defibrillator (ICD) devices, cardiac resynchronization therapy defibrillator devices, implantable monitoring or diagnostic devices, neurostimulators or other implantable devices or combinations thereof.

The IMD may include a lead that is directly connected to tissue to be affected by the IMD. The lead can include a tip portion that is directly connected to the anatomical tissue, such as a muscle bundle, and a lead body that connects to the device body or therapeutic driving device. It is generally known that the device body or case portion can be implanted in a selected portion of the anatomical structure, such as in a chest or abdominal wall, and the lead can be inserted through various venous portions so that the tip portion can be positioned at the selected position near or in the muscle group.

The IMD generally remains with the patient during the rest of the patient's natural life. The device itself may be changed or updated from time to time, however, the leads generally are not changed or replaced unless they become damaged. Thus, over time, the IMD can be exposed to various environmental factors. For example, the patient may undergo a magnetic resonance imaging (MRI) procedure or other high frequency imaging procedures. Magnetic resonance imaging has been developed as an imaging technique adapted to obtain both images of anatomical features of human patients as well as some aspects of the functional activities of biological tissue. These images have medical diagnostic value in determining the state of the health of the anatomical tissue examined.

In a magnetic-resonance imaging process, a patient is typically aligned to place the portion of the patient's anatomy to be examined in the imaging volume of the magnetic-resonance imaging apparatus. Such a magnetic-resonance imaging apparatus typically comprises a primary magnet for supplying a constant magnetic field which, by convention, is along the z-axis and is substantially homogeneous over the imaging volume and secondary magnets that can provide linear magnetic field gradients along each of three principal Cartesian axes in space. A magnetic field gradient refers to the variation of the field with respect to each of the three principal Cartesian axes.

The use of the magnetic-resonance imaging process with patients who have implanted medical devices, such as cardiac assist devices, often presents problems. As is known to those skilled in the art, implantable devices (such as implantable pulse generators and cardioverter/defibrillator/pacemakers) are sensitive to a variety of forms of electromagnetic interference because these enumerated devices include sensing and logic systems that respond to low-level electrical signals emanating from the monitored tissue region of the patient. Since the sensing systems and conductive elements of these implantable devices are responsive to changes in local electromagnetic fields, the implanted devices are vulnerable to external sources of severe electromagnetic noise, and in particular, to electromagnetic fields emitted during the magnetic resonance imaging procedure. Furthermore, patients with implantable devices are generally advised not to undergo magnetic resonance imaging procedures since one or more leads of the IMD may act as an antenna and have current or energy induced therein due to the MRI procedure. This induced current or energy can damage the IMD or anatomical tissue and cause injury to the patient. Accordingly, reduction or dissipation of the induced current or energy would be useful and beneficial to patients having an implanted medical device.

### SUMMARY

An implantable medical device (IMD) can include implantable pulse generator (IPG) devices, implantable cardioverter-defibrillators (ICD), cardiac resynchronization therapy defibrillator devices, neurostimulators, implantable monitoring devices or combinations thereof operating as a system comprising the device itself and one or more leads. In one example, the IMD may be an implantable cardiac device that can be positioned in a selected portion of the anatomical structure, such as a chest wall or abdominal wall, and a lead can be positioned through a vein or transvenously so that a lead tip can be implanted in a portion of the cardiac or heart muscle. Various portions of the IMD, such as a case or device body, or the lead(s) can be augmented according to this disclosure to include a piezoelectric transformer to reduce or eliminate detrimental affects to the IMD or the patient due to various external environmental factors.

According to the present disclosure, an implantable medical device is operable to sense signals from a patient and/or to provide a therapy to anatomical tissue of the patient in the presence of an interfering signal. The implanted medical device (IMD) includes at least one lead coupled at a proximal end to the implanted medical device and coupled at a distal end to the anatomical tissue and has a conductor therein coupled to electronic circuitry within the implanted medical device, e.g., sensing circuitry and/or therapy circuitry. A piezoelectric transformer is coupled in series between the conductor and the electrode near the distal end of the lead. As will be described in more detail below, an acoustic gap within the piezoelectric transformer prevents the interfering signal from damaging the electronic circuitry within the IMD or causing heating of the anatomical tissue that may injure the patient.

Also, a method disclosed wherein at least one lead is coupled at a proximal end to an implanted medical device and coupled at a distal end to anatomical tissue of a patient. The at least one lead has a conductor coupled to circuitry within the implanted medical device. A piezoelectric transformer is coupled in series between the conductor and an electrode of the at least one lead to permit the transmission of intended signals between the conductor and the electrode while preventing transmission of interfering signals generated during an MRI procedure from being transmitted between the conductor and the electrode.

Also disclosed is an implantable medical lead having a proximal end configured to couple to an implanted medical device and a distal end configured to couple to an anatomical tissue of a patient. A conductor extends along a length of the lead, wherein the conductor is configured to be coupled to electronic circuitry of the implanted medical device when the lead is coupled to the implantable medical device. A piezoelectric transformer is coupled in series between the conductor and an electrode near the distal end of the lead, wherein the piezoelectric transformer permits the transmission of intended signals between the electrode and the conductor while preventing interfering signals from being transmitted between the electrode and the conductor.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is an illustration of an IMD coupled to a human heart;

FIG. 2 is an illustration of an exemplary piezoelectric transformer suitable for use in the present disclosure;

FIG. 3 is an illustration of a piezoelectric transformer applied to a uni-polar lead according to one embodiment of the present disclosure;

FIG. 4 is an illustration of a piezoelectric transformer applied to a bi-polar lead according to one embodiment of the present disclosure;

FIG. 5 is an illustration of multi piezoelectric transformers applied to a lead according to one embodiment of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features. As indicated above, the present teachings are directed towards various embodiments of a piezoelectric transformer permitting the transmission of intended signals, such as physiologic signals or therapy signals, while preventing an interfering signal from being transmitted toward the circuitry for generating therapy or causing heating of the anatomical tissue. The interfering radiating field may, for example, be a radiating field generated by an MRI scanner. The techniques of this disclosure may, however, be used to reduce or eliminate the effect of other interfering radiating fields, such as interfering radiating fields generated by any medical or non-medical device.

In this application, the piezoelectric transformer as disclosed permits the transmission of intended signals, such as physiologic signals or therapy signals, while preventing an interfering signal from being transmitted toward the circuitry for generating therapy or causing heating of the anatomical tissue to protect the internal electronic circuitry of the IMD and/or the patient by reducing or eliminating detrimental affects to the IMD or the patient due to the interfering radiating field. For example, interfering radiating field may induce energy on one or more implantable leads coupled to the IMD. In some instances, the IMD inappropriately detects the induced energy on the leads as physiological signals, which may in turn cause the IMD to deliver undesired therapy or withhold desired therapy. This inappropriate detection is sometimes referred to as oversensing. In other instances, the induced energy on the leads result in the IMD not detecting physiological signals that are actually present, which may again result in the IMD delivering undesired therapy or withholding desired therapy.

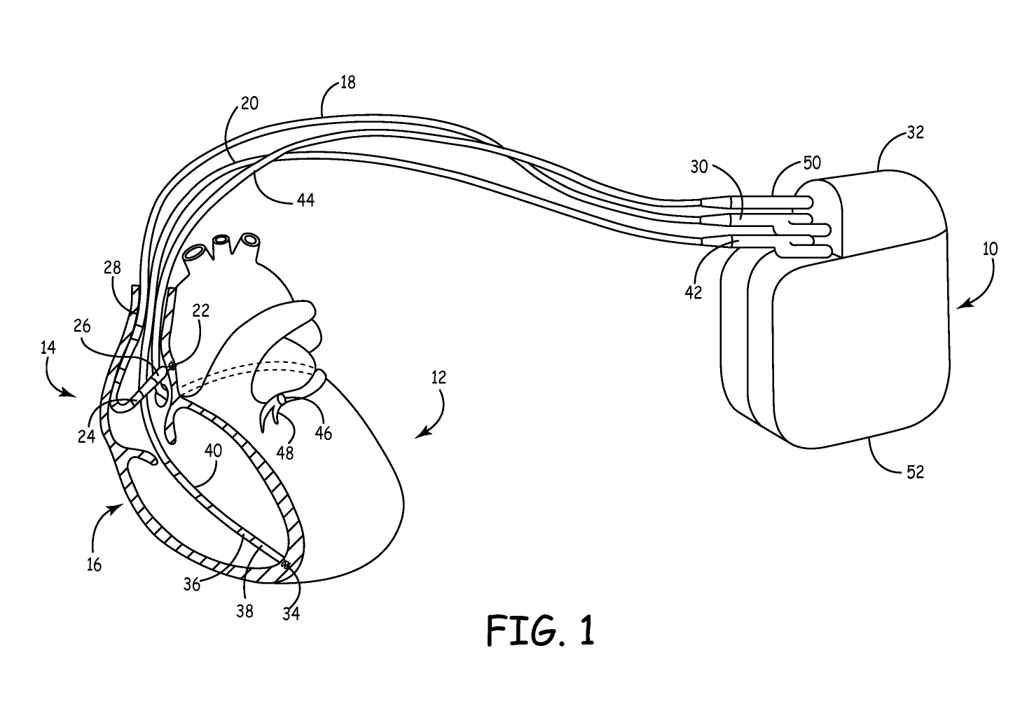

FIG. 1 depicts an exemplary implantable medical system that includes an IMD 10 and one or more leads extending from IMD 10 that may practice the techniques of this disclosure. In the example of FIG. 1, IMD 10 is an implantable multi-chamber pacemaker that includes cardioversion and defibrillation capability. In one preferred embodiment, techniques of this disclosure may be practiced by a device that paces a single cardiac chamber or several chambers, that paces one or more atria or one or more ventricles, and that paces in any of several pacing modes. However, the techniques of this disclosure is not limited to the particular IMD shown in FIG. 1, however, but may be practiced by any number of implantable devices, such as devices that provide stimulation therapy to other locations in the patient, including a stomach, brain, spinal cord, pelvic floor or other location in the patient. Moreover, the techniques of this disclosure are not limited to therapy delivery devices, but may also be practiced in monitoring or diagnostic devices, or devices that provide combinations of therapy, monitoring and diagnostics.

IMD 10 includes internal electronic circuitry. In one exemplary embodiment, IMD 10 includes therapy circuitry, e.g., an implantable pulse generator (IPG) that generates pacing stimuli or other electrical stimulation therapy to administer one or more therapies to heart 12. Pacing stimuli may be applied to the right atrium 14, for example, or the right ventricle 16, or both. IMD 10 also includes sensing circuitry to sense atrial and ventricular activations. In some instances, atrial and ventricular bipolar pace/sense electrode pairs at the distal ends of leads 18 and 20, respectively, carry out the pacing and sensing functions. In other instances, the pacing and sensing functions may be performed as unipolar, e.g., using one the electrodes located on the distal end of the respective lead and the housing or can of IMD 10 as the return electrode.

In right atrium 14, the distal end of atrial lead 18 includes an extendable helical, pace/sense tip electrode 22 and a pace/sense ring electrode 24. Helical electrode 22 extends from electrode head 26 into the atrial appendage. Pace/sense electrodes 22 and 24 are employed for atrial pacing and for sensing of P-waves indicative of atrial activation. The distal end of atrial lead 18 also includes an elongated coil defibrillation electrode 28 that can deliver a defibrillation shock to right atrium 14. Electrode 28 may also be used to deliver cardioversion therapy to right atrium 14.

Atrial lead 18 may include conductors that electrically couple electrodes 22, 24 and 28 to IMD 10. The conductors may be arranged coaxially, coradially, in parallel, or in another configuration, and may be insulated from one another and from the tissue of the patient. The proximal end of atrial lead 18 may include a bifurcated connector 30 that couples the conductors to a connector block 32 on IMD 10 and the conductors couple to internal electronic circuitry, including therapy and/or sensing circuitry, via feedthroughs.

In right ventricle 16, the distal end of ventricular lead 20 likewise may include a pace/sense tip electrode 34 and a pace/sense ring electrode 36. Pace/sense tip electrode 34 may be a helical electrode that extends from electrode head 38 toward the apex of heart 12. Pace/sense electrodes 34 and 36 are employed for ventricular pacing and for sensing of R-waves indicative of ventricular activation. The distal end of ventricular lead 20 also includes an elongated coil defibrillation electrode 40 that can deliver a defibrillation shock or cardioversion therapy to right ventricle 16.

Like atrial lead 18, ventricular lead 20 may include one or more insulated conductors that electrically couple electrodes 34, 36 and 40 to IMD 10. The proximal end of ventricular lead 20 may include a bifurcated connector 42 that couples the conductors to connector block 32 and the conductors couple to internal electronic circuitry via feedthroughs.

FIG. 1 also illustrates deployment of a coronary sinus lead 44. Coronary sinus lead 44 may include one or more insulated conductors. The proximal end of coronary sinus lead 44 may include one or more electrodes, such as pace/sense electrode 46. Pace/sense electrode 46 may be deployed within the great vein 48 of heart 12, and may be used to deliver pacing therapies to the left side of heart 12. A connector 50 at the proximal end of the coronary sinus lead 44 couples the conductors in lead 44 to connector block 32 and the conductors couple to internal circuitry via feed-through (not shown). In some embodiments, coronary sinus lead 44 may include an elongated exposed coil wire defibrillation electrode (not shown).

IMD 10 includes a housing 52 that may serve as a “can” electrode. In unipolar pacing operations, IMD 10 may deliver an electrical stimulation to heart 12 via an electrode disposed on one or more of leads 18, 20 or 44, with housing 52 being a part of the return current path. In bipolar pacing operation, by contrast, IMD 10 may deliver an electrical stimulation to heart 12 via a tip electrode, with a ring electrode providing the principal return current path. IMD 10 may also perform unipolar sensing using the using an electrode disposed on one or more of leads 18, 20 or 44 in conjunction with housing 52. In some embodiments, housing 52 includes two or more electrodes, and IMD 10 may detect electrical signals generated by heart 12 with the two or more electrodes disposed in housing 52.

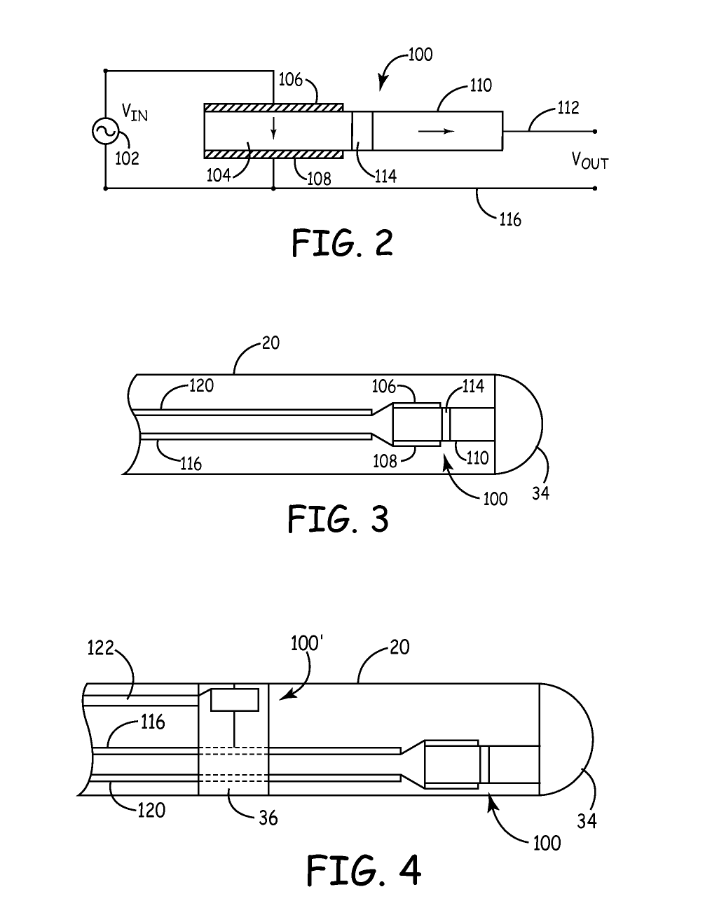

FIG. 2 illustrates an exemplary piezoelectric transformer 100 suitable for use with the present disclosure. An input circuit 102 drives piezoelectric transformer 100 with an input signal V_{IN} having a frequency matched approximately to the resonant frequency of piezoelectric transformer 100. In one embodiment, the input circuit 102 may be an electrical stimulation generator (e.g., pacing circuitry) of IMD 10 and the input signal comprises pacing pulses to pace the heart of a patient. Piezoelectric transformer 100 includes a first (input) resonator 104 sandwiched between conductive plates 106, 108, and a second (output) resonator 110 having an output 112 that generates an output signal V_{OUT}. Second resonator 110 may also be sandwiched between conductive plates, e.g., similar to conductive plates 106, 108 of first resonator 104. The output 112 may then be electrically coupled to the non-grounded conductive plate. Those skilled in the art will appreciate that the positioning and location of the conductive plates 106, 108 and output 112 may be determined by optimized piezo structure determined by form factor, optimal piezoelectric vibration mode (e.g., transverse, thickness or radial) and the locations shown are for ease of illustration. A gap 114 separates resonator 104 from resonator 110. Gap 114 may be filled with an acoustically transmissive gel as is known in the art, so the acoustic energy transfer is maximized. One such acoustically transmissive gel suitable for use in conjunction with the present disclosure is Aquasonic Ultrasound Gel by Parker Laboratories, however, any other similar acoustically transmissive gel may be used. Those skilled in the art will appreciate that the gap width of gap 114 will be determined by the operating frequency, mechanical robustness, conductive plate placement and the ability to block the MRI induced voltage expected for any particular lead/electrode combination. A common ground 116 serves as reference for input signal V_{IN} and output signal V_{OUT}. Common ground 116 may be a ground conductor that extends along a length of the lead from IMD 10 to the distal end of the lead and couple to conductive plate 108 resonator 104 and/or a corresponding grounded conductive plate of resonator 110. Alternatively, common ground 116 may be a ground to the tissue, e.g., via a ring electrode or other electrode near the distal end of the lead.

As is known in the acoustic arts, acoustic (piezoelectric) transducers operate to cause an electrical signal to be applied by conductive plates 106 and 108 causing a shape change in the resonator 104. The shape change causes mechanical distortions that pass through the gel filled gap 114 to the resonator 110, where the process is reversed and mechanical distortions produce electrical signals at electrode 112. Thus, as described herein, piezoelectric transformer 100 serves to transmit intended signals, e.g., therapeutic energy from the IMD to anatomical tissue of a patient or sensed signals from the anatomical tissue to the IMD. However, interfering energy fields, such as those associated with magnetic resonant imaging (MRI) equipment, cannot cross the gap 114 provided that the piezoelectric transformer 100 is not tuned to the resonant frequency of the MRI signals (e.g., 64 MHz in the case of a 1 T MRI device or 128 MHz in the case of a 3 T MRI device). In this way, any MRI energy picked up by the lead (which may act as an antenna to the MRI signals) cannot reach the patient's anatomical tissue and cause localized heating that may be injurious to the patient.

Piezoelectric transformer 100 offers a small size and low profile, facilitating placement of the piezoelectric transformer within an implantable lead. In addition, piezoelectric transformer 100 offers good power efficiency. For example, some commercially available piezoelectric transformers are known to offer 80 to 90 percent power efficiency.

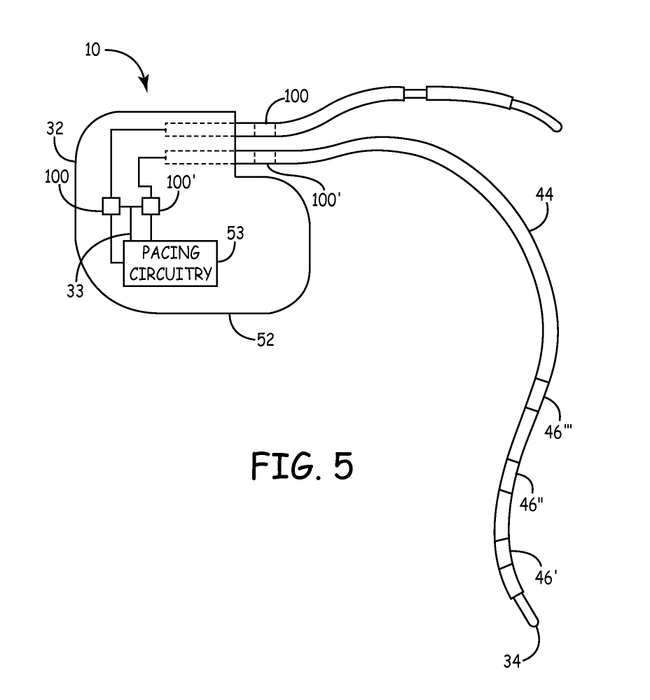

FIG. 3 illustrates one embodiment of the present disclosure applied to a uni-polar ventricular lead 20 as discussed in conjunction with FIG. 1 (however, it will be appreciated that the present disclosure applies also to atrial leads, coronary sinus leads, neruo stimulation leads, spinal leads, uni-polar, bi-polar, multi-polar or any other implantable lead for delivery of therapy). As can be seen, the piezoelectric transformer 100 is coupled on an input end to a conductor 120 (and ground 116) that delivers electrical signals to and from the IMD 10. The piezoelectric transformer 100 is configured such that intended signals may cross the gap 114, i.e., intended signals may be transmitted between electrode 34 and the conductor 120. For example, a therapeutic energy delivered by therapy circuitry of IMD 10 can cross the gap 114 to be delivered to a patient's anatomical tissue via the tip electrode 34 as described in detail above with respect to FIG. 2. However, as described above, electromagnetic interfering (EMI) energy, such as MRI signals, cannot cross the gap 114 and cannot be transmitted to the patient's anatomical tissue to cause localized heating. This is because interfering energy fields, such as those associated with magnetic resonant imaging (MRI) equipment, cannot cross the gap 114 provided that the piezoelectric transformer 100 is not tuned to the resonant frequency of the MRI signal (typically, 64 MHz in the case of a 1.5 Tesla MRI device and 128 MHz in the case of a 3 Tesla MRI device). Thus, the filtering of unwanted signal energy is accomplished by the DC blocking of the acoustic transformer. That is, any incident signal energy that gives rise to a DC voltage component on the lead due to clipping, clamping or IMD internal circuitry loading is blocked from passing to the secondary side of the acoustic transformer, and from there to the electrode(s) (e.g., tip electrode or ring electrode). As discussed below in conjunction with FIG. 5, an acoustic transformer located near the proximal side of the lead or within the IMD housing would provide a DC block and AC filter to the IMD internal circuitry.

FIG. 4 illustrates one embodiment of the present disclosure applied a uni-polar ventricular lead 20 as discussed in conjunction with FIG. 1 (again, however, it will be appreciated that the present disclosure applies also to atrial leads, coronary sinus leads, neruo stimulation leads, spinal leads, uni-polar, bi-polar, multi-polar or any other implantable lead for delivery of therapy). As can be seen, in addition to the piezoelectric transformer 100 coupled to the tip electrode 34, a second piezoelectric transformer 100′ is coupled to the ring electrode 36. However, electromagnetic interfering (EMI) energy, such as MRI signals, cannot cross the gap 114 of piezoelectric transformer 100′ to the patient's anatomical tissue to cause localized heating. Additionally, since the EMI energy cannot cross gap 114, it cannot cause the IMD 10 to inappropriately detect induced energy on the leads as physiological signals, which may in turn cause the IMD 10 to deliver undesired therapy or withhold desired therapy. Thus, the IMD 10 is protected from damage and the patient is protected from both tissue damage and inappropriate therapies.

In some instances, lead 20 may not include second piezoelectric transformer 100′ coupled to ring electrode 36. Instead, conductor 122 may directly couple to ring electrode 36. In this case, ring electrode 36 may sense physiological signals and conduct the sensed signals to sensing circuitry within IMD 10 via conductor 122 to assist in the delivery of therapy to the patient or for monitoring a condition of the patient. In another example, lead 20 may include the second piezoelectric transformer 100′ coupled to ring electrode 36, but may include a second ring electrode that is not coupled to piezoelectric transformer for sensing physiological signals.

FIG. 5 illustrates one embodiment of the present disclosure applied to a uni-polar coronary sinus lead 44 as discussed in conjunction with FIG. 1 (again, however, it will be appreciated that the present disclosure applies also to atrial leads, ventricular leads, neruo stimulation leads, spinal leads or any other implantable lead for delivery of therapy). Coronary sinus leads may be used to sense and deliver therapy to the left ventricle of a patient's heart. Typically, the best position for the electrodes are determined empirically after implantation. Accordingly, coronary sinus lead 44 has several ring electrodes 46′, 46″ and 46′″ along the length of the lead and a tip electrode 34 near the distal end of the lead. Often, the ring electrode is used for therapy delivery as well as sensing. Thus, one of the ring electrodes can be selected after implantation or multiple ring electrodes may be active to achieve the best depolarization of the left ventricle. In one embodiment, the multiple ring electrodes are coupled to a common conductor and are multiplexed (e.g., TDM, FDM or QPM) to be separately addressable by the IMD 10 and can be activated, deactivated or have its function changed from sensing to therapy delivery. They may also be addressable by having a different resonant frequency or respond to a different signal burst or digital code. Each ring electrode 46′, 46″ and 46″ and the tip electrode 34 may have a piezoelectric transformer 100 (not shown in FIG. 5) coupled to it and functions as discussed above to permit the therapy or sensing information to cross the gap 114, while preventing electromagnetic interfering (EMI) energy, such as MRI signals, from crossing the gap 114. In this way, the interfering energy cannot be transmitted to the circuitry within the IMD 10 or to the patient's anatomical tissue to cause localized heating. Additionally, since the EMI energy cannot cross the gap, it cannot cause the IMD to inappropriately detect induced energy on the leads as physiological signals, which may in turn cause the IMD to deliver undesired therapy or withhold desired therapy. Thus, the IMD is protected from damage and the patient is protected from both tissue damage and inappropriate therapies.

FIG. 5 also illustrates another use for the piezoelectric transformer 100 of the present disclosure useful for the situation where EMI energy is picked up at the middle or near the proximal end of a lead. As shown, piezoelectric transformers 100 and 100′ can be positioned within the housing 52 to prevent EMI energy from being conducted through the leads and connector block 32 so that the pacing circuitry 53 is protected from damage. In one embodiment, the piezoelectric transformers 100 and 100′ are coupled to a common ground 33, however, if the housing 52 is used as the common potential in the system, the piezoelectric transformers 100 and 100′ would be coupled to the housing 52. Alternately, the piezoelectric transformers 100 and 100′ can be positioned near the proximal end of the lead as illustrated by the dashed lines in FIG. 5.

While specific examples have been described in the specification and illustrated in the drawings, it will be understood by those of ordinary skill in the art that various changes may be made and equivalents may be substituted for elements thereof without departing from the scope of the present disclosure as defined in the claims. Furthermore, the mixing and matching of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. Therefore, it is intended that the present disclosure not be limited to the particular examples illustrated by the drawings and described in the specification as the best mode presently contemplated for carrying out this disclosure, but that the scope of the present disclosure will include any embodiments falling within the foregoing description and the appended claims.

1. An implantable medical system comprising:
an implantable medical device having electronic circuitry therein; and
at least one lead coupled at a proximal end to the implanted medical device and coupled at a distal end to the anatomical tissue, the lead including:
a conductor coupled at the proximal end of the lead to the electronic circuitry within the implanted medical device;
an electrode near the distal end of the lead; and
a piezoelectric transformer coupled in series between the conductor and the electrode near the distal end of the lead;
wherein the piezoelectric transformer permits the transmission of intended signals between the electrode and the conductor while preventing interfering signals from being transmitted between the electrode and the conductor.

2. The implantable medical system of claim 1, wherein the interfering signal comprises a magnetic or electric field generated during a magnetic resonance imaging (MRI) scan.

3. The implantable medical system of claim 1, wherein the electronic circuitry includes therapy circuitry and the intended signals comprise therapy signals generated by the therapy circuitry.

4. The implantable medical system of claim 1, wherein the electronic circuitry includes sensing circuitry and the intended signals comprise signals sensed by the electrode for transmission to the sensing circuitry.

5. The implantable medical system of claim 1, wherein the conductor comprises a first conductor, the electrode comprises a first electrode, and the piezoelectric transformer comprises a first piezoelectric transformer, the lead further comprising:
a second conductor coupled at the proximal end of the lead to the electronic circuitry within the implanted medical device;
a second electrode near the distal end of the lead;
a second piezoelectric transformer that is coupled in series between the second conductor and the second electrode, wherein the second piezoelectric transformer permits the transmission of intended signals between the second electrode and the second conductor while preventing interfering signals from being transmitted between the second electrode and the second conductor.

6. The implantable medical system of claim 1, wherein the piezoelectric transformer is located near the distal end of the lead.

7. The implantable medical system of claim 6, further comprising a second piezoelectric transformer located near a proximal end of the lead.

8. The implantable medical system of claim 1, further comprising a second piezoelectric transformer within the implantable medical device and coupled in series between the conductor of the lead and the electronic circuitry of the implantable medical device.

9. The implantable medical system of claim 1, wherein the piezoelectric transformer comprises:
a first resonator coupled to the conductor and a ground conductor;
a second resonator coupled to the tip electrode; and
a gap between the first and second resonator;
wherein the first and second resonators are tuned to a resonant frequency of other than 64 MHz or 128 MHz.

10. The implantable medical system of claim 1, wherein the interfering signal has a frequency of 64 MHz and is generated during a magnetic resonance imaging (MRI) scan.

11. The implantable medical system of claim 1, wherein the interfering signal has a frequency of 128 MHz and is generated during a magnetic resonance imaging (MRI) scan.

12. A method comprising:
providing at least one lead coupled at a proximal end to an implanted medical device and coupled at a distal end to anatomical tissue of a patient and having a conductor therein coupled to circuitry within the implanted medical device; and
providing a piezoelectric transformer coupled in series between the conductor and an electrode of the lead to permit the transmission of intended signals between the conductor and the electrode while preventing transmission of interfering signals generated during an MRI procedure from being transmitted between the conductor and the electrode.

13. The method of claim 12, which includes the step of providing a second piezoelectric transformer coupled in series between a second conductor coupled to the medical device and a second electrode near the distal end of the lead to permit the transmission of intended signals between the second conductor and the second electrode while preventing transmission of interfering signals generated during an MRI procedure from being transmitted between the second conductor and the second electrode.

14. The method of claim 12, wherein providing the piezoelectric transformer comprises providing the piezoelectric transformer near the distal end of the lead.

15. An implantable medical lead comprising
a proximal end configured to couple to an implanted medical device;
a distal end configured to couple to an anatomical tissue of a patient;
a conductor extending along a length of the lead, wherein the conductor is configured to be coupled to electronic circuitry of the implanted medical device when the lead is coupled to the implantable medical device;
an electrode near the distal end of the lead; and
a piezoelectric transformer coupled in series between the conductor and the electrode near the distal end of the lead, wherein the piezoelectric transformer permits the transmission of intended signals between the electrode and the conductor while preventing interfering signals from being transmitted between the electrode and the conductor.

16. The implantable medical lead of claim 15, wherein the intended signals comprise at least one of therapy signals to be delivered by the electrode and physiological signals sensed by the electrode.

17. The implantable medical lead of claim 15, wherein the conductor comprises a first conductor, the electrode comprises a first electrode, and the piezoelectric transformer comprises a first piezoelectric transformer, the lead further comprising:
a second conductor extending along a length of the lead, wherein the second conductor is configured to be coupled to the electronic circuitry of the implanted medical device when the lead is coupled to the implantable medical device;
a second electrode near the distal end of the lead;
a second piezoelectric transformer that is coupled in series between the second conductor and the second electrode, wherein the second piezoelectric transformer permits the transmission of intended signals between the second electrode and the second conductor while preventing interfering signals from being transmitted between the second electrode and the second conductor.

18. The implantable medical lead of claim 15, wherein the piezoelectric transformer is located near the distal end of the lead.

19. The implantable medical lead of claim 18, further comprising a second piezoelectric transformer located near a proximal end of the lead.

20. The implantable medical lead of claim 15, wherein the piezoelectric transformer comprises:
a first resonator coupled to the conductor and a ground conductor;
a second resonator coupled to the tip electrode; and
a gap between the first and second resonator;
wherein the first and second resonators are tuned to a resonant frequency other than 64 MHz or 128 MHz.
